(19) United States
(12) Patent Application Publication
(10) Pub. No.: US 2021/0379310 A1
(43) Pub. Date: Dec. 9, 2021

(54) CONNECTOR FOR MEDICATION DISPENSER

(71) Applicant: Wei ZHANG, Irvine, CA (US)

(72) Inventor: Wei ZHANG, Irvine, CA (US)

(21) Appl. No.: 17/342,223

(22) Filed: Jun. 8, 2021

Related U.S. Application Data
(60) Provisional application No. 63/036,334, filed on Jun. 8, 2020.

(51) Int. Cl. A61M 16/04; A61M 16/08

(52) U.S. Cl. CPC A61M 16/0477; A61M 16/0841; A61M 16/0463

(57) ABSTRACT

A connector a medication dispenser includes a first portion coupling the connector to the medication dispenser and a second portion configured to couple the connector to a breathing apparatus. The first portion has a first opening with a first shape and a first size. The second portion has a second opening with a second shape and a second size that are different from the first shape and the first size of the first opening, respectively.

### CROSS-REFERENCE TO RELATED APPLICATION

The present disclosure claims priority to U.S. provisional Application No. 63/036,334 filed on Jun. 8, 2020, which is incorporated by reference herein for all purposes.

### BACKGROUND

The present disclosure relates to a connector for a medication dispenser (e.g., an aerosol dispenser), a medication applicator (e.g., an aerosol applicator) including the connector and the medication dispenser, and a breathing system including the medication applicator and a breathing apparatus (e.g., an endotracheal tube, a breathing circuit, or the like).

A quick medication delivery to a patient with accurate dosage during a medical procedure can be lifesaving. For example, anesthesiologist often use an aerosol dispenser to treat intraoperative airway spasms. However, because an entrance of an endotracheal tube (ETT) does not match the aerosol dispenser, these two ends cannot make a firm seal when they are put together, resulting in aerosol leakage and making it difficult to precisely control the dosage delivered to the patient as well as wasting the medication.

### SUMMARY

Embodiments of the present application relate to a connector for a medication dispenser (e.g., an aerosol dispenser), a medication applicator (e.g., an aerosol applicator) including the connector and the medication dispenser, and a breathing system including the medication applicator and a breathing apparatus (e.g., an endotracheal tube, a breathing circuit, or the like).

In an embodiment, a connector for a medication dispenser includes a first portion coupling the connector to the medication dispenser and a second portion coupling the connector to a breathing apparatus. The first portion has a first opening with a first shape and a first size. The second portion has a second opening with a second shape and a second size that are different from the first shape and the first size of the first opening, respectively.

In an embodiment, a medication applicator includes a medication dispenser and a connector configured to couple the medication dispenser to a breathing apparatus. The connector includes a first portion coupling the connector to the medication dispenser and a second portion coupling the connector to a breathing apparatus. The first portion has a first opening with a first shape and a first size. The second portion has a second opening with a second shape and a second size that are different from the first shape and the first size of the first opening, respectively.

In an embodiment, a breathing system includes a breathing apparatus and a connector configured to couple a medication dispenser to the breathing apparatus. The connector includes a first portion coupling the connector to the medication dispenser and a second portion coupling the connector to the breathing apparatus. The first portion has a first opening with a first shape and a first size. The second portion has a second opening with a second shape and a second size that are different from the first shape and the first size of the first opening, respectively.

### BRIEF DESCRIPTION OF THE DRAWINGS

FIG. 1 illustrates a breathing system according to an embodiment of the present disclosure.

FIG. 2 illustrates a breathing system according to an embodiment of the present disclosure.

FIGS. 3A, 3B, 3C, and 3D illustrate a side view of an aerosol dispenser, a side view of a connector, a cross-sectional view of the connector, and a side view of an adaptor, respectively, according to embodiments of the present disclosure.

FIG. 4 illustrates a breathing system according to an embodiment of the present disclosure.

FIGS. 5A, 5B, 5C, 5D, and 5E illustrate a top view, a rear view, a side view, a front view, and a bottom view of a medication applicator (e.g., aerosol applicator), respectively, according to an embodiment of the present disclosure.

FIGS. 6A, 6B, 6C, and 6D illustrate a rear view, a side view, a front view, and a cross-sectional view of a connector, respectively, according to an embodiment of the present disclosure.

### DETAILED DESCRIPTION

Embodiments of the present application relate to a connector for a medication dispenser (e.g., an aerosol dispenser), a medication applicator (e.g., an aerosol applicator) including the connector and the medication dispenser, and a breathing system including the medication applicator and a breathing apparatus (e.g., an endotracheal tube, a breathing circuit, or the like).

In an embodiment, a connector for a medication dispenser includes a first portion coupling the connector to a mouthpiece of an aerosol dispenser and a second portion configured to couple the connector to an adaptor of an ETT. The first portion has a first opening with a substantially rectangular shape matching that of the mouthpiece, and the second portion has a second opening with a circular shape matching that of the adaptor of the ETT. As a result, the connector can make firm seals with the mouthpiece and the adaptor, thereby substantially preventing aerosol leakage, precisely controlling the dosage delivered to a patient, and saving the medication.

In an embodiment, a connector for a medication dispenser includes a first portion coupling the connector to a mouthpiece of an aerosol dispenser and a second portion coupling the connector to a sampling port of a breathing circuit. The first portion has a first opening with a substantially rectangular shape matching that of the mouthpiece, and the second portion has a second opening with a circular shape matching that of an opening of the sampling port. The connector further includes a third portion coupling the connector to the sampling port together with the second portion through Lure lock fitting. As a result, the connector can make firm seals with the mouthpiece and the sampling port, thereby substantially preventing aerosol leakage, precisely controlling the dosage delivered to a patient, and saving the medication. In addition, the connector may be coupled to the sampling port of the breathing circuit without removing the breathing circuit from an ETT, and thus a medical procedure may be performed safely without worrying potential contamination from a patient.

A detailed description of embodiments is provided below along with accompanying figures. The scope of this disclosure is limited only by the claims and encompasses numerous alternatives, modifications and equivalents. Although steps of various processes are presented in a given order, embodiments are not necessarily limited to being performed in the listed order. In some embodiments, certain operations may be performed simultaneously, in an order other than the described order, or not performed at all.

Numerous specific details are set forth in the following description. These details are provided to promote a thorough understanding of the scope of this disclosure by way of specific examples, and embodiments may be practiced according to the claims without some of these specific details. Accordingly, the specific embodiments of this disclosure are illustrative, and are not intended to be exclusive or limiting. For the purpose of clarity, technical material that is known in the technical fields related to this disclosure has not been described in detail so that the disclosure is not unnecessarily obscured.

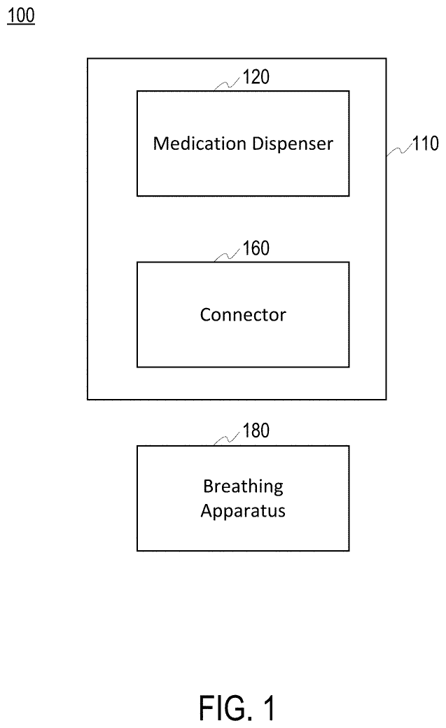

FIG. 1 illustrates a breathing system 100 according to an embodiment of the present disclosure. The breathing system 100 may include a medication applicator 110 and a breathing apparatus 180.

The mediation applicator (e.g., an aerosol applicator) 110 may include a medication dispenser 120 and a connector 160. In an embodiment, the medication dispenser 120 is an aerosol dispenser and may provide one or more aerosol medications stored therein to the breathing apparatus 180 through the connector 160.

The connector 160 may include a first portion coupling the connector 160 to the medication dispenser 120 and a second portion coupling the connector 160 to the breathing apparatus 180. The first portion and the second portion of the connector 160 may form firm seals with the medication dispenser 120 and the breathing apparatus 180, respectively, thereby substantially preventing aerosol leakage, precisely controlling the dosage delivered to a patient, and saving the medication.

The breathing apparatus 180 may couple the connector 160 to the patient. In an embodiment, the breathing apparatus 10 is an ETT, a breathing circuit, or the like.

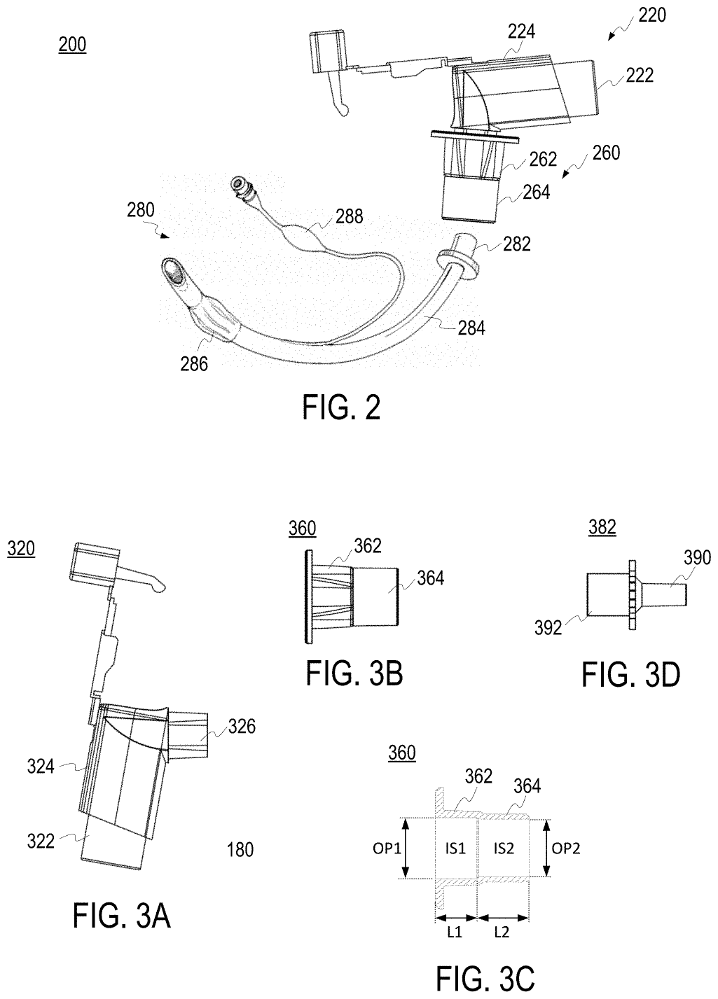

FIG. 2 illustrates a breathing system 200 according to an embodiment of the present disclosure.

Referring to FIG. 2, the breathing system 200 may include a medication dispenser (e.g., an aerosol dispenser) 220, a connector 260, and an endotracheal tube (ETT) 280. The dispenser 220 may include a container 222 and a casing 224. The container 222 may store one or more aerosol medications to address medical issues occurring during a medical procedure. For example, the container 22 may store albuterol or atrovent to relieve airway spasm occurring during anesthesia.

The casing 224 may be fixedly coupled to the container 224 by wrapping a portion of the container 222. The casing 224 may include a mouthpiece (e.g., a mouthpiece 326 in FIG. 3A) for delivering the aerosol medication from the container 222.

The connector 260 may couple the mouthpiece of the casing 224 to a portion (e.g., the adaptor 282) of the ETT 280 to form respective seals at both ends of the connector 260. In an embodiment, the connector 260 includes a first portion 262 and a second portion 264, such that the first portion 262 may be coupled to the mouthpiece to form a first seal, and the second portion 264 may be coupled to the adaptor 282 to form a second seal.

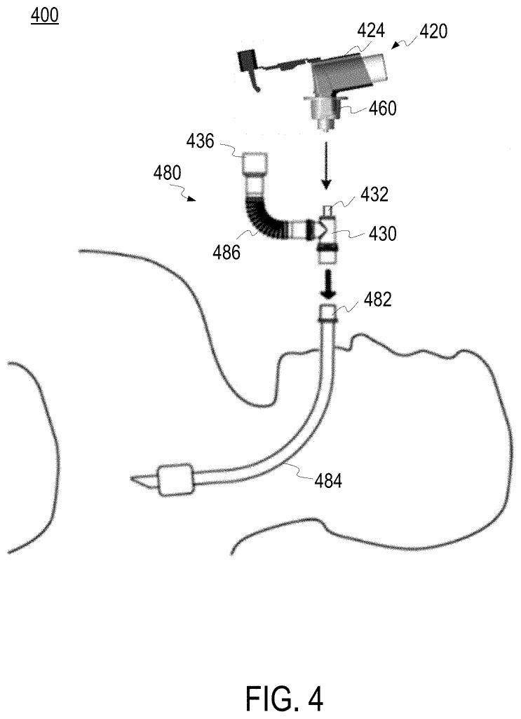

The ETT 280 may include the adaptor 282, a tubing 284, a balloon 288, and a cuff 286. For example, the adaptor 282 may couple the ETT 280 to a portion of a breathing circuit (e.g., a breathing circuit 480 in FIG. 4).

FIGS. 3A, 3B, 3C, and 3D respectively illustrate a side view of an aerosol dispenser 320, a side view of a connector 360, and a cross-sectional view of the connector 360, and a side view of an adaptor 382, according to embodiments of the present disclosure. For example, the cross-sectional view of the connector 360 in FIG. 3C may be obtained by cutting the connector 360 with a plane passing through a vertical centerline of the connector 360 when seen in a rear view of the connector 360.

Referring to FIGS. 3A to 3D, the aerosol dispenser 320 includes a mouthpiece 362 and the connector 360 includes a first portion 362 and a second portion 364. The mouthpiece 326 may be inserted into a first portion 362 of the connector 360 to form a first seal, and the adaptor 382 may be inserted into a second portion 364 of the connector 360 to form a second seal. For example, the first portion 362 of the connector 360 may have a first inner space IS1 to accommodate a portion of the mouthpiece 326 to form the first seal and the second portion 364 of the connector 360 may have a second inner space IS2 to accommodate a portion of the adaptor 382.

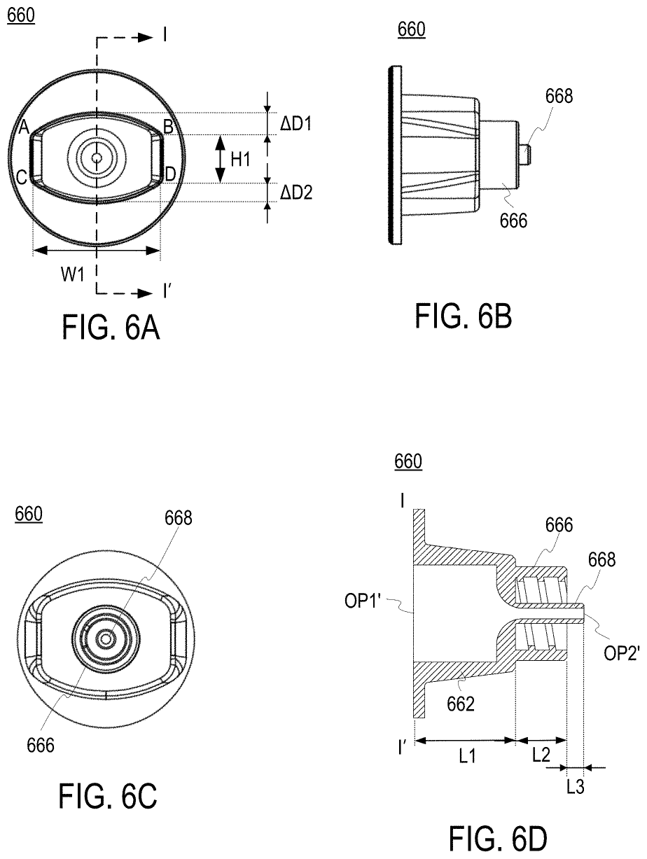

The first portion 362 of the connector 360 may have a first opening OP1 corresponding to an outer perimeter of the mouthpiece 326. In an embodiment, the first opening OP1 of the first portion 362 may have a substantially rectangular shape corresponding to that of an outer surface of the mouthpiece 326, similarly to that of a first opening OP1′ of a connector 660 shown in FIG. 6A.

The first portion 362 of the connector 360 may have a first length L1 along a first direction (e.g., an axial direction) that is perpendicular to the first opening OP1. In an embodiment, the first length L1 of the first portion 362 of the connector 360 may be in a range from 15 mm to 21 mm.

In an embodiment, the second portion 364 of the connector 360 may have a second opening OP2 corresponding to an outer perimeter of the adaptor 382. For example, the second opening OP2 of the second portion 364 may have a circular shape corresponding to that of an outer surface of a first portion 392 of the adaptor 382.

The second portion 364 of the connector 360 may have a second length L2 along a first direction (e.g., an axial direction) that is perpendicular to the second opening OP2. In an embodiment, the second length L2 of the second portion 364 may be in a range from 15 mm to 21 mm.

The first length L1 and the second length L2 may be sufficiently long (e.g., each being equal to or longer than 15 mm) to ensure securing the inserted mouthpiece 362 and the inserted portion 392 of the adaptor 382, respectively. When a flow of the aerosol medication flows in the first direction (e.g., the axial direction), the flow expands in a second direction (e.g., a radial direction) perpendicular to the first direction. As a result, when the sum of the first length L1 and the second length L2 is excessively long, an outer portion of the medication flow may be significantly interrupted by one or more of an inner surface of the inserted mouthpiece 326, inner surfaces of the first portion 362 and the second portion 364 of the connector 360, and an inner surface of the inserted portion 392 of the adaptor 382. Accordingly, the sum of the first length L1 and the second length L2 may be sufficiently short (e.g., equal to or shorter than 42 mm) to effectively transport the aerosol medication through the connector 360.

The first portion 392 of the adaptor 382 functioning as an entrance of an ETT (e.g., the ETT 280 in FIG. 2) may not match the mouthpiece 326 of the aerosol dispenser 320. For example, the first portion 392 of the adaptor 382 has a circular cross-section, whereas the mouthpiece 326 has a substantially rectangular cross-section. Thus, ends of the first portion 392 and the mouthpiece 326 cannot make a firm seal when they are put together, resulting in aerosol leakage and making it difficult to precisely control the dosage delivered to the patient as well as wasting the medication.

The connector 360 according to an embodiment of the present disclosure may have the first opening OP1 and the second opening OP2 that differ in shape. For example, the connector 360 may have the first opening OP1 matching the substantially rectangular cross-section of the mouthpiece 326 and the second opening OP2 matching the circular cross-section of the first portion 392 of the adaptor 382. As a result, the connector 360 can make firm seals with the mouthpiece 326 and the adaptor 382, thereby substantially preventing aerosol leakage, precisely controlling the dosage delivered to the patient, and saving the medication. The first opening OP1 and the second opening OP2 also may differ in size. For example, a first area of the first opening OP1 may be in a range from 250 mm² to 425 mm², and a second area of the second opening OP2 may be in a range from 160 mm² to 240 mm². For example, a ratio of the first area of the first opening OP1 over the second area of the second opening OP1 may be in a range from 1.1 to 2.7.

FIG. 4 illustrates a breathing system 400 according to an embodiment of the present disclosure. The breathing system 400 includes a medication dispenser (e.g., an aerosol dispenser) 420, a connector 460, and a breathing circuit 480. The aerosol dispenser 420 may have structures and functions similar to those of the aerosol dispenser 220 in FIG. 2, and thus detailed descriptions thereof may be omitted for the interest of brevity.

The breathing circuit 480 includes a first adaptor 430, a sampling port 432, a hose 486, and a second adaptor 436. The first adaptor 430 may be coupled to an adaptor 482 of an ETT. Although not shown in FIG. 4, the breathing circuit 480 may further include other elements such as a Y-piece coupled to the second adaptor 436, breathing hoses coupled to the Y-piece, and valves coupled to the breathing hoses.

The connector 460 may couple a mouthpiece of a casing 424 of the medication dispenser 420 to a sampling port 432 of the breathing circuit 480. In an embodiment, the connector 460 may be coupled to the sampling port 460 through Lure lock fitting. Although the breathing circuit 480 shown in the embodiment of FIG. 4 includes the sampling port 432 attached to the first adaptor 430, embodiments of the present disclosure are not limited thereto. For example, the breathing circuit 480 may include two or more sampling ports (not shown) and these sampling ports may be attached to various parts of the breathing circuit 480.

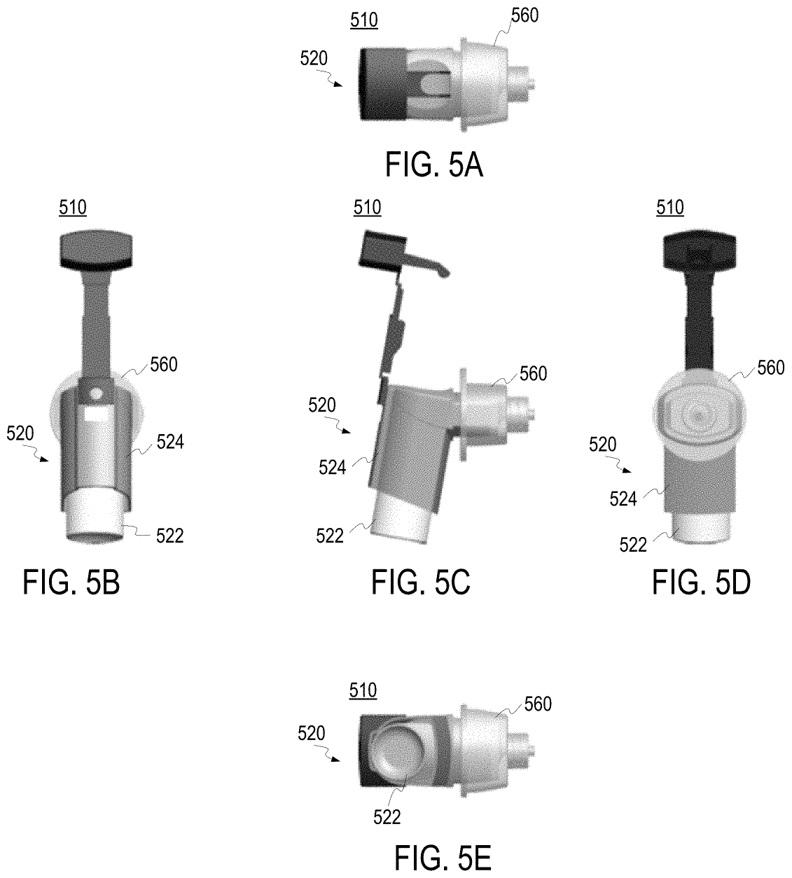

FIGS. 5A, 5B, 5C, 5D, and 5E respectively illustrate a top view, a rear view, a side view, a front view, and a bottom view of a medication applicator (e.g., aerosol applicator) 510 according to an embodiment of the present disclosure. The aerosol applicator 510 may include a connector 560 and an aerosol dispenser 520 that are suitable for use as the connector 460 and the aerosol dispenser 420 shown in FIG. 4, respectively.

FIGS. 6A, 6B, 6C, and 6D respectively illustrate a rear view, a side view, a front view, and a cross-sectional view of a connector 660, according to an embodiment of the present disclosure. The connector 660 may be suitable for use as the connector 460 shown in FIG. 4.

The connector 660 may include a first portion 662, a second portion 668, and a third portion 666. An aerosol dispenser (e.g., the aerosol dispenser 420 in FIG. 4) may include a mouthpiece to be inserted into the first portion 662 of the connector 660 to form a first seal, and a sampling port (e.g., the sampling port 432) of a breathing circuit (e.g., the breathing circuit 480 in FIG. 4) may be inserted into a space between the second portion 668 and the third portion 666 of the connector 660 to form a second seal.

The first portion 662 of the connector 660 may have a first opening OP1′ corresponding to an outer perimeter of the mouthpiece. In an embodiment, the first opening OP1′ of the first portion 662 may have a substantially rectangular shape corresponding to that of an outer surface of the mouthpiece. For example, with respect to the orientation shown in FIG. 6A, the first opening OP1′ has a bulged rectangular shape with two straight left and right edges and two curved top and bottom edges. Each of the curved edges may be convex such that a maximum deviation ΔD1 or ΔD2 from a horizontal line AB or CD connecting a corresponding pair of vertices of the first opening OP1′ is equal to or less than 40% of a length H1 of each of the left and right edges.

The first portion 662 of the connector 660 may have a first length L1 along a first direction (e.g., an axial direction) that is perpendicular to the first opening OP1′. In an embodiment, the first length L1 of the first portion 662 of the connector 660 may be in a range from 15 mm to 21 mm.

The second portion 668 and the third portion 666 of the connector 660 may be coupled to the sampling port. In an embodiment, the second portion 668 and the third portion 666 of the connector 660 may be coupled to the sampling port through Lure lock fitting. For example, an outer surface of the second portion 668 of the connector 660 may be inserted into an opening of the sampling port, and an inner surface of the third portion 666 of the connector 660 may be threadedly coupled to an outer surface of the sampling port, thereby forming a firm seal to substantially prevent aerosol leakage. Referring back to FIG. 4, the connector 460 according to an embodiment of the present disclosure may be coupled to the sampling port 432 of the breathing circuit 480 without removing the breathing circuit 480 from the ETT. Accordingly, a medical procedure may be performed safely without worrying potential contamination from a patient.

The third portion 666 of the connector 660 may have a second length L2 along a first direction (e.g., an axial direction) that is perpendicular to the second opening OP2′. In an embodiment, the second length L2 of the third portion 666 may be in a range from 4 mm to 12 mm.

The second portion 668 of the connector 660 may protrude from the third portion 666 in the axial direction by a third length L3. In an embodiment, the third length L3 may be in a range from 1.5 mm to 3.5 mm.

The connector 660 according to an embodiment of the present disclosure may have the first opening OP1′ and the second opening OP2′ that differ in shape. For example, the connector 660 may have the first opening OP1′ matching the substantially rectangular cross-section of the mouthpiece of the aerosol dispenser and the second opening OP2′ matching a portion of the circular cross-section of the opening of the sampling port. Thus, the connector 660 can make firm seals with the mouthpiece and the sampling port, thereby substantially preventing aerosol leakage, precisely controlling the dosage delivered to the patient, and saving the medication. The first opening OP1 and the second opening OP2 also may differ in size. For example, a first area of the first opening OP1′ may be in a range from 250 mm² to 425 mm², and a second area of the second opening OP2′ may be in a range from 2 mm² to 5 mm². For example, a ratio of the first area of the first opening OP1′ over the second area of the second opening OP1′ may be in a range from 50 to 200.

Aspects of the present disclosure have been described in conjunction with the specific embodiments thereof that are proposed as examples. Numerous alternatives, modifications, and variations to the embodiments as set forth herein may be made without departing from the scope of the claims set forth below. Accordingly, embodiments as set forth herein are intended to be illustrative and not limiting.

What is claimed is:

1. A connector for a medication dispenser, comprising:
a first portion configured to couple the connector to the medication dispenser, the first portion having a first opening with a first shape and a first size; and
a second portion configured to couple the connector to a breathing apparatus, the second portion having a second opening with a second shape and a second size that are different from the first shape and the first size of the first opening, respectively.

2. The connector of claim 1, wherein the first shape of the first opening is a substantially rectangular shape, and the second shape of the second opening is a circular shape.

3. The connector of claim 2, wherein the medication dispenser is an aerosol dispenser, the breathing apparatus is an endotracheal tube (ETT), the first portion of the connector is coupled to a mouthpiece of the aerosol dispenser, and the second portion of the connector is coupled to an adaptor of the ETT.

4. The connector of claim 3, wherein a ratio of the first area of the first opening over the second area of the second opening is in a range from 1.1 to 2.7.

5. The connector of claim 3, wherein the first portion has a first length in a specific direction perpendicular to the first opening, and the second portion has a second length in the specification direction, and
wherein each of the first length and the second length is in a range from 15 mm to 21 mm.

6. The connector of claim 2, wherein the substantially rectangular shape has a first straight edge, a second straight edge facing the first straight edge, and a curved edge, the curved edge meeting the first straight edge at a first vertex and meeting the second straight edge at a second vertex, a maximum deviation of the curved edge from a line connecting the first vertex and the second vertex being equal to or less than 40% of a length of each of the first and second straight edges.

7. The connector of claim 2, wherein the medication dispenser is an aerosol dispenser, the breathing apparatus is a breathing circuit, the first portion of the connector is coupled to a mouthpiece of the aerosol dispenser, and the second portion of the connector is coupled to a sampling port of the breathing circuit.

8. The connector of claim 7, a ratio of the first area of the first opening over the second area of the second opening is in a range from 50 to 200.

9. The connector of claim 8, further comprising a third portion, the third portion and the second portion being configured to couple the connector to the sampling port through Lure lock fitting.

10. The connector of claim 9, wherein an outer surface of the second portion of the connector is configured to be inserted into an opening of the sampling port of the breathing circuit, and an inner surface of the third portion of the connector is configured to be threadedly coupled to an outer surface of the sampling port of the breathing circuit.

11. The connector of claim 9, wherein the first portion has a first length in a specific direction perpendicular to the first opening, the second portion has a second length in the specification direction, and
wherein the first length of the first portion is in a range from 15 mm to 21 mm, and the second length of the second portion is in a range from 4 to 12 mm.

12. The connector of claim 11, wherein the second portion protrudes from the third portion in the specific direction by a third length, the third length being in a range from 2 mm to 4 mm.

13. A medication applicator, comprising:
a medication dispenser; and
a connector configured to couple the medication dispenser to a breathing apparatus, wherein the connector comprises:
a first portion configured to couple the connector to the medication dispenser, the first portion having a first opening with a first shape and a first size; and
a second portion configured to couple the connector to the breathing apparatus, the second portion having a second opening with a second shape and a second size that are different from the first shape and the first size of the first opening, respectively.

14. The applicator of claim 13, wherein the first shape of the first opening is a substantially rectangular shape, and the second shape of the second opening is a circular shape.

15. The applicator of claim 14, wherein the medication dispenser is an aerosol dispenser, the breathing apparatus is an endotracheal tube (ETT), the first portion of the connector is coupled to a mouthpiece of the aerosol dispenser, and the second portion of the connector is coupled to an adaptor of the ETT.

16. The applicator of claim 14, wherein the medication dispenser is an aerosol dispenser, the breathing apparatus is a breathing circuit, the first portion of the connector is coupled to a mouthpiece of the aerosol dispenser, and the second portion of the connector is coupled to a sampling port of the breathing circuit.

17. A breathing system, comprising:
a breathing apparatus;
a connector configured to couple a medication dispenser to the breathing apparatus, wherein the connector comprises:
a first portion configured to couple the connector to the medication dispenser, the first portion having a first opening with a first shape and a first size; and
a second portion configured to couple the connector to the breathing apparatus, the second portion having a second opening with a second shape and a second size that are different from the first shape and the first size of the first opening, respectively.

18. The system of claim 17, wherein the first shape of the first opening is a substantially rectangular shape, and the second shape of the second opening is a circular shape.

19. The system of claim 18, wherein the breathing apparatus is an endotracheal tube (ETT), the medication dispenser is an aerosol dispenser, the first portion of the connector is coupled to a mouthpiece of the aerosol dispenser, and the second portion of the connector is coupled to an adaptor of the ETT.

20. The system of claim 18, wherein the breathing apparatus is a breathing circuit, the medication dispenser is an aerosol dispenser, the first portion of the connector is coupled to a mouthpiece of the aerosol dispenser, and the second portion of the connector is coupled to a sampling port of the breathing circuit.
